(19) United States
(12) Patent Application Publication
(10) Pub. No.: US 2020/0143942 A1
(43) Pub. Date: May 7, 2020

(54) BIOLOGICAL INFORMATION MONITORING SYSTEM, BIOLOGICAL INFORMATION MONITORING METHOD, AND BIOLOGICAL INFORMATION MONITORING PROGRAM

(71) Applicant: Fujita Medical Instruments Co., Ltd., Tokyo (JP)

(72) Inventors: Hironobu MAEDA, Tokyo (JP); Shougo TANAKA, Tokyo (JP)

(73) Assignee: Fujita Medical Instruments Co., Ltd., Tokyo (JP)

(21) Appl. No.: 16/737,157

(22) Filed: Jan. 8, 2020

Related U.S. Application Data
(63) Continuation of application No. PCT/JP2018/027569, filed on Jul. 23, 2018.

(30) Foreign Application Priority Data
Jul. 24, 2017 (JP) 2017-142727
Jun. 26, 2018 (JP) 2018-120942

(51) Int. Cl. G16H 40/67; H04W 4/80; H04W 4/38; A61B 5/00; A61B 5/1455; A61B 5/022; A61B 5/024; A61B 5/1172; A61B 5/1171; G16H 15/00; G16H 10/60; G06F 3/0482; H04L 29/08

(52) U.S. Cl. CPC G16H 40/67; H04L 67/1097; H04W 4/38; A61B 5/002; A61B 5/742; A61B 5/14553; A61B 5/0006; A61B 5/022; A61B 5/02416; A61B 5/1172; A61B 5/1176; A61B 5/1171; G16H 15/00; G16H 10/60; G06F 3/0482; H04W 4/80

(57) ABSTRACT

To provide a biological information monitoring system, a biological information monitoring method, and a biological information monitoring program for monitoring biological information of a plurality of subjects by a simple method. A biometric information monitoring system includes a plurality of measuring devices which are attached to a plurality of subjects to measure biometric information of the subjects, and a portable information processing terminal for an observer, for displaying the biometric information of the subjects measured by the measuring devices. In response to a transmission command transmitted from a terminal communication unit, a device communication unit transmits to the terminal communication unit the biometric information of one subject to which a plurality of the measuring devices storing one piece of identification information are attached. A processing unit generates display information for displaying on a display screen of the information processing terminal the biometric information received by the terminal communication unit.

### CROSS-REFERENCE TO RELATED APPLICATIONS

This application is a continuation of PCT International Patent Application No. PCT/JP2018/027569, filed Jul. 23, 2018, which claims priority to Japanese Patent Application Nos. 2018-120942 filed Jun. 26, 2018 and 2017-142727 filed Jul. 24, 2017, the content of each of which is incorporated herein by reference in its entirety.

### BACKGROUND

### 1. Technical Field

The present invention relates to a biological information monitoring system, a biological information monitoring method, and a biological information monitoring program. More specifically, the present invention relates to a biological information monitoring system including a plurality of measuring devices attached to a plurality of subjects for measuring the biological information of the subjects; and a portable information processing terminal for an observer for displaying the biological information of the subjects measured by the measuring devices, a biological information monitoring method performed in the monitoring system, and a biological information monitoring program for embodying the monitoring system.

### 2. Description of Related Art

Conventionally, a central monitor installed at a nurse station in a medical institution has been known as a biological information monitoring apparatus for monitoring states of a plurality of patients. For example, as shown in Patent Document 1, the central monitor is configured to receive biological information (for example, electrocardiogram, blood pressure, and arterial oxygen saturation) of each patient from a bedside monitor installed on the bedside of each patient in an intensive care unit or a sickroom, and display it on the screen. In the same manner, Patent Document 2 discloses an invention for suppressing a decrease in utilization efficiency of a basic screen of the central monitor.

Further, in relation to the bedside monitor, Patent Document 3 discloses a technique for a nurse to check the biological information of the patients on a display screen of a portable information processing terminal.

Specifically, the technique disclosed in Patent Document 3 discloses a technique in which an ID tag such as a wristband is attached to the patient as a means for identifying the patient, and the nurse stands near the subject patient lying on the bed, and acquires identification information directly from the ID tag of the target patient.

### PRIOR ART DOCUMENTS

### Patent Documents

[Patent documents 1]

Japanese Laid-Open Patent Publication No. 2005-124903A

[Patent documents 2]

Japanese Laid-Open Patent Publication No. 2015-000177A

[Patent documents 3]

Japanese Laid-Open Patent Publication No. 2016-123573A

### SUMMARY OF THE INVENTION

### Problem(s) to be Solved by the Invention

However, when a plurality of subjects from which biometric information is to be acquired are present, collection of the biometric information by the conventional method is considerably complicated. In addition, when a plurality of measuring devices is attached to each of a plurality of subjects, it is expected that the complexity of collecting the biological information is further increased.

Accordingly, an object of the present invention is to provide a biological information monitoring system, a biological information monitoring method, and a biological information monitoring program for collecting and monitoring biological information of a plurality of subjects by a simple method.

### Means for Solving the Problem(s)

A biological information monitoring system according to an embodiment of the present invention includes a plurality of measuring devices attached to a plurality of subjects for measuring biological information of the subjects, and a portable information processing terminal for an observer for displaying the biological information of the subjects measured by the measuring devices. Each measuring device includes: a measuring unit, a device storage, an identification information generator, and a device communication unit. The measuring unit is configured to be managed in advance as a set together with other measuring devices that are attached to one subject, and measure biological information of the subject. The device storage is configured to store the identification information given for each set of the measuring devices, and the biological information measured by the measuring unit. The identification information generator is configured to issue additional information in response to a request, generate identification information including the additional information and the identification information as identification information of one subject to which the measuring devices included in one set are attached, and allow the device storage to store the identification information. The device communication unit is configured to transmit the identification information of the subject stored by the device storage to the information processing terminal. The information processing terminal includes: a terminal communication unit, a processing unit, and a selection receiving unit. The terminal communication unit is configured to receive a plurality of pieces of the identification information of the subjects transmitted from the device communication unit of each of the measuring devices attached to the subjects. The processing unit is configured to generate display information for displaying the pieces of identification information of the subjects to allow the observer to select them. The selection receiving unit is configured to receive a selection of one piece of identification information of one subject from the pieces of identification information of the subjects by the observer. The terminal communication unit transmits a command to each device communication unit for allowing the device communication unit to transmit biological information of the one subject to which the measuring devices storing the one piece of identification information of the one subject received by the selection receiving unit are attached. The device communication unit transmits, to the terminal communication unit, the biological information of the one subject to which the measuring devices storing the one piece of identification information of the one subject are attached in response to the transmission command transmitted from the terminal communication unit. The processing unit generates display information for displaying the biological information received by the terminal communication unit on a display screen of the information processing terminal.

### Advantage of the Invention

The biological information monitoring system, biological information monitoring method, and biological information monitoring program of the present invention allow biological information of a plurality of subjects to be monitored by a simple method.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a conceptual diagram illustrating an example of an image of a biological information monitoring system according to an embodiment of the present invention,

FIG. 2 is a configuration diagram illustrating an example of a configuration of a biological information monitoring system according to an embodiment of the present invention,

FIGS. 3A and 3B are configuration diagrams illustrating examples of data configuration according to an embodiment of the present invention,

FIG. 4 is a diagram illustrating an example of a display screen according to an embodiment of the present invention,

FIG. 5 is a diagram illustrating an example of a display screen according to an embodiment of the present invention,

FIG. 6 is a configuration diagram illustrating an example of a configuration of a biological information monitoring system according to an embodiment of the present invention,

FIG. 7 is a configuration diagram illustrating an example of a data configuration according to an embodiment of the present invention,

FIG. 8 is a conceptual diagram illustrating an example of an image of the biological information monitoring system according to an embodiment of the present invention,

FIG. 9 is a conceptual diagram illustrating an image of a biological information monitoring system according to an embodiment of the present invention at a disaster site,

FIG. 10 is a flowchart illustrating a flow of a biological information monitoring method according to an embodiment of the present invention, and

FIG. 11 is a configuration diagram illustrating a functional configuration of a biological information monitoring program according to an embodiment of the present invention.

### DETAILED DESCRIPTION OF EMBODIMENTS

The biological information monitoring system, biological information monitoring method, and biological information monitoring program according to an embodiment of the present invention will be described in detail below with reference to the drawings. In the following description, the same components are described using the same reference numerals.

<Biological Information Monitoring System>

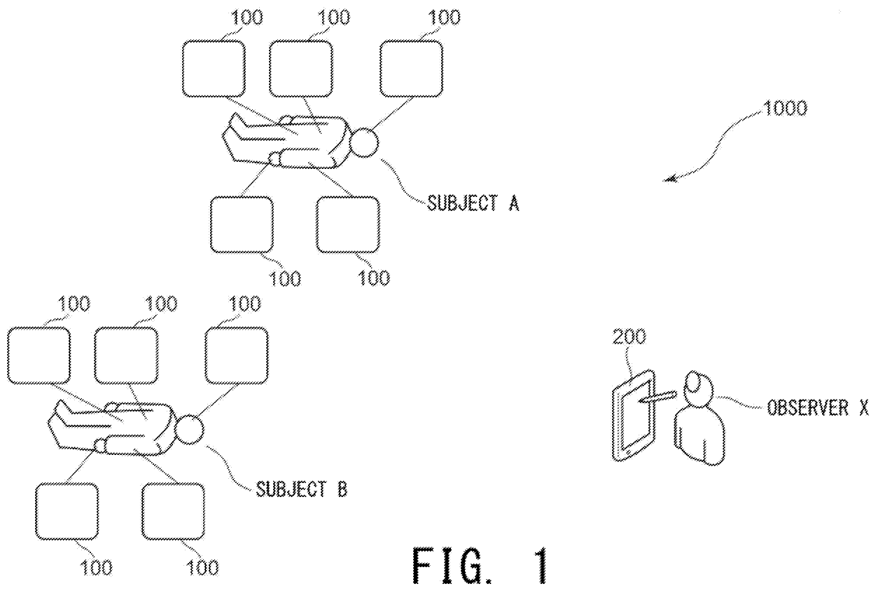

FIG. 1 is a conceptual diagram illustrating an image of a biological information monitoring system 1000 according to an embodiment of the present invention. As shown in FIG. 1, the biological information monitoring system 1000 includes a plurality of measuring devices 100 attached to a plurality of subjects A and B for measuring biological information of the subjects A and B, and a portable information processing terminal 200 for an observer X for displaying the biological information of the subjects.

Although FIG. 1 shows only two subjects A and B as a plurality of subjects, the number of subjects is not especially limited. In the same manner, in FIG. 1, although only five measuring devices 100 are shown as a plurality of measuring devices 100 attached to one subject, the number of measuring devices 100 is not especially limited. Various measuring devices 100 may be attached as needed.

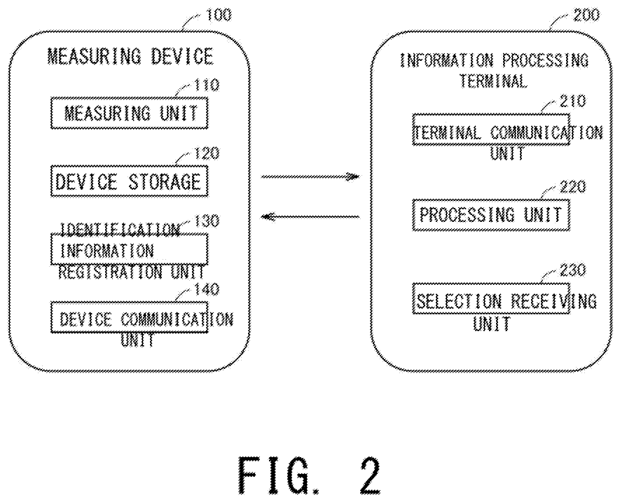

FIG. 2 is a block diagram illustrating details of the configuration of portions of the biological information monitoring system 1000 according to an embodiment of the present invention.

<<Measuring Devices>>

As shown in FIG. 2, the measuring devices 100 each include a measuring unit 110, a device storage 120, an identification information registration unit 130, and a device communication unit 140.

The measuring unit 110 measures biological information of a subject.

The biological information here is directed to vital signs of the subject, and includes information measured by the measuring devices 100 such as an intracerebral oxygen saturation monitor, an electrocardiograph, a sphygmomanometer, a pulse oximeter, and an echo, for example.

The device storage 120 stores identification information for identifying the subject and the biological information measured by the measuring unit 110.

The identification information here includes information for identifying the subject, and the identification of the subject may be performed directly or indirectly. The method for identification above will be described below.

Further, the identification information can be specifically an identification number indicated by a numeral or alphabet, for example. Moreover, it is preferable that the identification information be provided so that a plurality of measuring devices 100 attached to one subject is identified as one set.

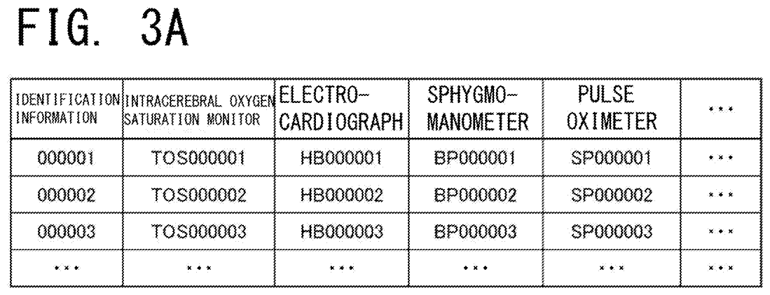

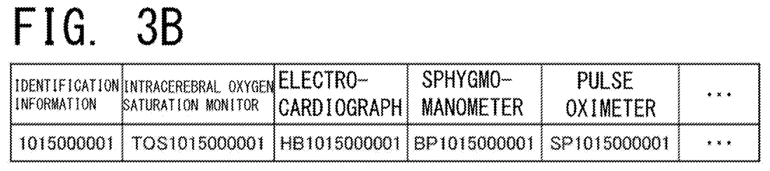

FIG. 3A is a data configuration diagram illustrating a data configuration related to the identification information stored in the device storage 120. As shown in FIG. 3, for each of the measuring devices 100 included in the set to which the identification number “000001” is assigned, device identification numbers with alphabets such as “TOS” indicating a brain oxygen saturation monitor, “HB” indicating an electrocardiograph, “BP” indicating a sphygmomanometer, and “SP” indicating a pulse oximeter are assigned to the head of the identification number “000001” in advance. In this case, the set of the measuring devices 100 can be identified as the set of measuring devices 100 having the device identification number “000001,” and the type of each of the measuring devices 100 can also be identified.

The identification information registration unit 130 issues additional information in response to the registration request. Moreover, the identification information generator 130 generates identification information including the additional information and the identification information as identification information of one subject to which the measuring devices 100 included in one set are attached. Furthermore, the identification information generator 130 causes the device storage 120 to store the generated identification information of the one subject.

The registration request used to issue the additional information is generated, for example, when each measuring device 100 is attached to the subject or when each measuring device 100 is operated after the measuring device 100 is attached to the subject. The additional information is directed to information that specifies the situation, such as time information and position information. Although a description is given below assuming that the additional information is directed to the time information, it may be directed to, in place of the time information, the position information, date/time information including the date/time in the time, or a combination of the time information and the position information. This additional information is included in the registration request by the information processing terminal 200, for example.

FIG. 3B illustrates an example of the identification information of the subject generated by the identification information generator 130 when the identification information described with reference to FIG. 3A is set in the device storage 120 of each measuring device 100. For example, when the time information “10:15” is included in the registration request, the additional information “1015” is issued based on the time information, and the identification information “000001” is combined with the additional information to be identification information of the subject. Further, the identification information obtained by adding the device identification number of the device to this becomes the identification information of each measuring device 100. This generates identification information that identifies the subject based on the additional information and the identification information (device identification number) set for each set of the devices, and causes each device to store the generated information. Moreover, the identification information can be used as the only identification information in the world.

The identification information registration unit 130 may delete the identification information of one subject stored in the device storage 120 in response to the termination request. In addition, the identification information registration unit 130 issues new additional information again in response to a new registration request, and newly generates the identification information of the subject using the new additional information. The identification information registration unit 130 then causes the device storage 120 to store the newly generated identification information of the subject. At this time, if the identification information of the previous subject is stored in the device storage 120, the information is updated to the newly generated identification information of the subject.

The device communication unit 140 transmits the identification information stored in the device storage 120 to the information processing terminal 200.

This transmission can be performed using wireless communication, for example, and a specific example of the communication means will be described later.

<<Information Processing Terminal>>

The information processing terminal 200 includes a terminal communication unit 210, a processing unit 220, and a selection receiving unit 230. The information processing terminal 200 is directed to a portable terminal, and includes, for example, a touch panel having an input function and an output function (not shown). In addition, the information processing terminal may include a camera or microphone serving as an input function and a speaker serving as an output function.

The terminal communication unit 210 receives a plurality of pieces of identification information transmitted from the device communication units 130 of the measuring devices 100 attached to the subjects.

The processing unit 220 generates display information for displaying a plurality of pieces of identification information to allow the observer to select them.

The selection receiving unit 230 receives selection of one piece of a plurality of pieces of identification information by the observer.

The terminal communication unit 210 then transmits a command to the device communication unit 130 for causing the device communication unit 130 to transmit the biological information of the one subject to which the measuring devices 100 storing one piece of the identification information received by the selection receiving unit 230 are attached.

The device communication unit 130 transmits, to the terminal communication unit 210, the biological information of the one subject to which the measuring devices storing the one piece of identification information are attached in response to the transmission command transmitted from the terminal communication unit 210.

The processing unit 220 generates display information for displaying the biological information received by the terminal communication unit 210 on the display screen of the information processing terminal 200.

According to the configuration above, only a simple operation in which the observer selects the one piece of identification information from a plurality of pieces of identification information displayed on the display screen of the portable information processing device allows the observer to collect the biological information of the one subject to which the measuring devices storing the one piece of identification information are attached and monitor it on the display screen.

The identification information stored in the measuring devices attached to the one subject can be directed to identification information that identifies that the measuring devices are attached to the one subject. Details of the identification information are as described above.

Information exchange between the measuring devices and the information processing terminal can be performed using Bluetooth (registered trademark) low energy BLE wireless communication technology.

The BLE wireless communication technology is directed to wireless communication using Bluetooth (registered trademark) low energy BLE. The use of the communication technology, for example, allows about 256 measuring devices to be connected in an identifiable range of about 10 m. The maximum number of simultaneous connections depends on the performance of the BLE device, and thus the number is not limited to them.

The processing unit 220 generates display information for displaying the biological information associated with the one piece of identification information selected by the observer within one display screen.

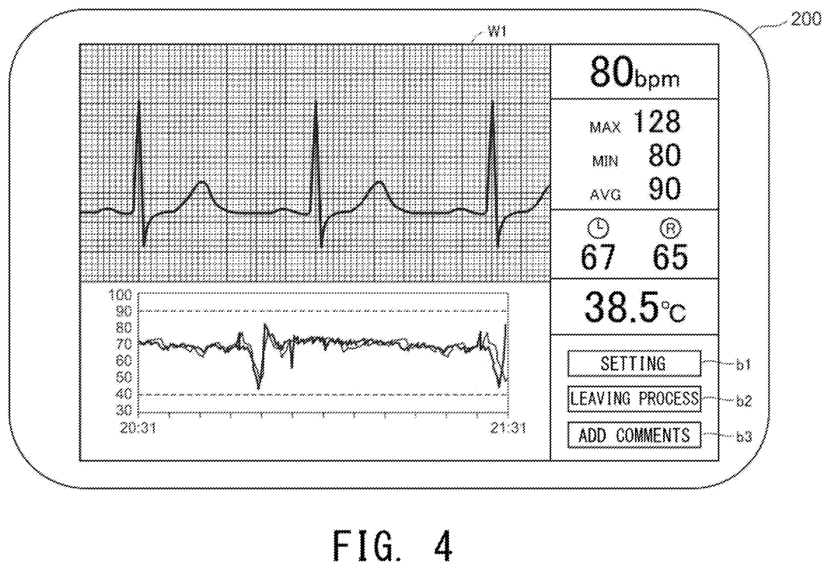

FIG. 4 illustrates an example of a display screen W1 that displays biometric information associated with the one piece of identification information selected by the observer. As shown in FIG. 4, a plurality of pieces of biological information associated with the one piece of identification information can be displayed on one screen on the display screen W1. In addition, the display screen W1 includes a setting button b1, a leaving process button b2, and a comment addition button b3. The setting button b1 is used when each measuring device 100 is attached to the subject and measurement is started. The leaving processing button b2 is used when the measuring device 100 is removed from the subject and the measurement is completed. The comment addition button b3 is used when the observer adds some comments regarding the measurement. When the comment addition button b3 is manipulated, a comment input screen (not shown) is displayed by the processing unit 220. In addition, the comments input to the comment input screen by the observer are stored in the storage (not shown) of the information processing terminal 200, for example.

The processing unit 220 displays a plurality of pieces of identification information to allow the observer to select them, and also displays information about the measuring devices 100 associated with the pieces of identification information in a selectable manner.

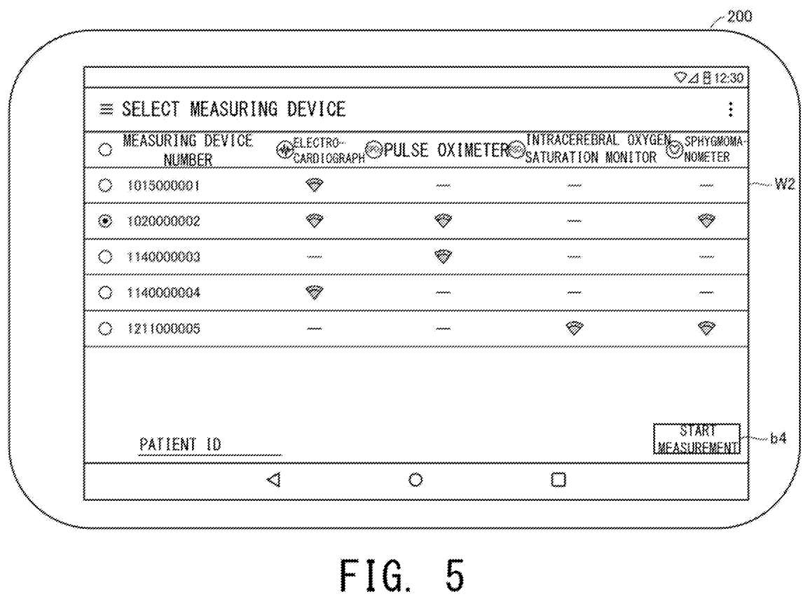

FIG. 5 illustrates an example of a display screen W2 that displays a plurality of pieces of identification information and information about a plurality of measuring devices in a selectable manner. As shown in FIG. 5, a plurality of pieces of identification information are displayed on the display screen W2 to allow the observer to select them, and information about a plurality of measuring devices 100 are also displayed in a selectable manner.

The information about the measuring devices can be directed to information indicating that the information on the devices is received. At this time, the selection of this icon allows the screen to display only the biometric information from this device so that it can be checked.

As shown in FIG. 5, the processing unit 220 generates display information for displaying the instruction button b4 for receiving the instruction from the observer on the display screen W2, which instruction button b4 is provided for allowing the device communication unit 140 to transmit, to the terminal communication unit 210, the biometric information of one subject to which the measuring devices 100 storing one piece of identification information are attached in response to the transmission command transmitted from the terminal communication unit 210.

<Modification 1>

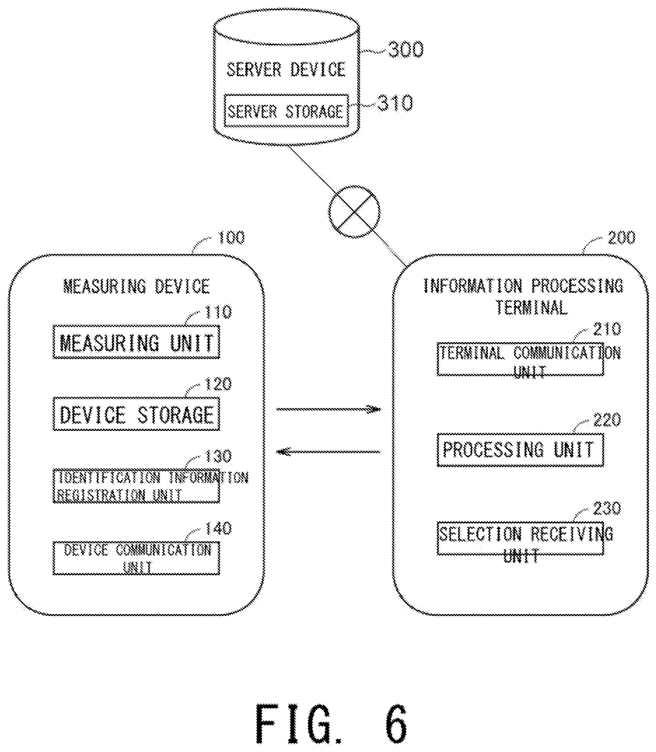

As shown in FIG. 6, the information processing terminal 200 may be connected through a network to the server device 300.

The server device 300 includes a server storage 310. The configuration of the server 300 is not especially limited. The server 300 may implement communication with the information processing terminal 200 using any communication protocol.

The server storage 310 associates and stores the identification information with the subject to which the measuring devices 100 storing the identification information are attached, and the processing unit 220 generates display information for displaying the subjects associated with a plurality of corresponding pieces of identification information received by the terminal communication unit 210 to allow the observer to select the subjects in place of the identification information.

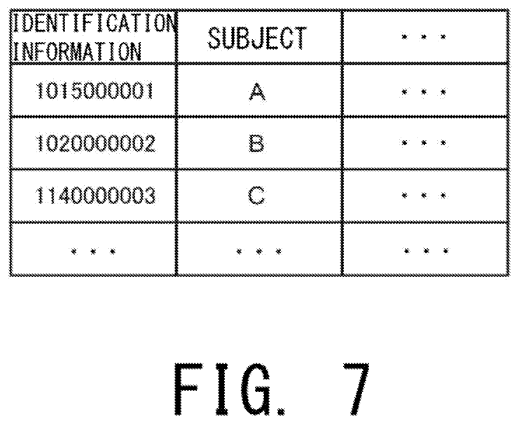

FIG. 7 is a diagram illustrating an example of a data configuration stored in the server storage 310. Storing the data as shown in FIG. 7 allows the observer to associate and recognize the identification information with the name of the subject. In addition, although it is preferable for the subject to indicate his/her identification number or name in a state or means allowing the observer to observe it, the means is not especially limited to this. An electromagnetic means may substitute for the means without requiring the observation by the observer.

The measuring devices 100 may be two or more measuring devices selected from a brain oxygen saturation monitor, an electrocardiograph, a blood pressure monitor, a pulse oximeter, and an echo.

Even when a new device is added to a set of measuring devices prepared in advance, the new device must have identification information corresponding to the set so that the observation is allowed without requiring any new settings.

<Modification 2>

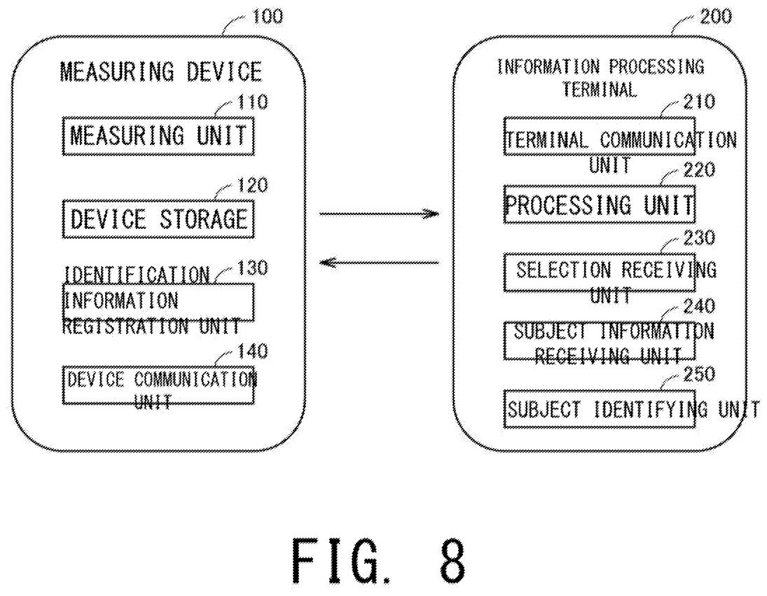

As illustrated in FIG. 8, the information processing terminal 200 further includes a subject information receiving unit 240 and a subject identifying unit 250.

The subject information receiving unit 240 receives subject information of the subject acquired from the subject by the observer.

Although the subject information may include at least one of name information, fingerprint information, vein information, face information, and/or iris information, the information is not limited thereto. The information may be directed to any information that can identify a subject.

In addition, the subject information received by the subject information receiving unit 240 is not limited to one, and a plurality of pieces of subject information may be received. This increases the precision that identifies the subject.

The subject identifying unit 250 specifies one subject having subject information corresponding to the subject information received by the subject information receiving unit 240 from the subject information database stored in advance in a predetermined storage.

The predetermined storage may be included in the information processing terminal 200, or may be included in an external device that can be connected to the information processing terminal by wire/wireless.

The subject information database stores the subject information above in advance. Specifically, when the measuring devices 100 are attached to the subject, it is preferable that the name information, fingerprint information, vein information, face information, and/or iris information of the subject, for example, be acquired and stored in the storage as the subject information database, The subject information can be acquired by using a dedicated device, or using a camera included in the information processing terminal 200, for example. The name information can also be acquired by input using a touch panel provided in the information processing terminal 200 or by voice recognition technology.

The terminal communication unit 210 then transmits a command to the device communication unit 130 for causing the device communication unit 130 to transmit the biological information of one subject to which a plurality of measuring devices storing one piece of identification information received by the selection receiving unit 230 is attached, or biological information of one subject identified by the subject identifying unit 250.

The configuration allows the observer to automatically identify the subject by using the information processing terminal 200 as long as the subject information of the subject is acquired without needing to identify the identification information of the subject by the observer himself/herself.

The subject information may include at least one piece of the name information, fingerprint information, vein information, face information, and/or iris information. The subject information can be acquired by using a dedicated device, or using a camera included in the information processing terminal 200, for example. The name information can also be acquired by input using a touch panel provided in the information processing terminal 200 or by voice recognition technology.

In addition, the biological information monitoring system 1000 according to an embodiment of the present invention further includes an information processing device 400 for remotely monitoring the biological information.

The information processing device 400 is directed to a device for monitoring the biological information of the subject remotely from a medical institution such as a hospital, and the device 400 is not especially limited to this as long as it can access a web page via the Internet.

The information processing terminal 200 and the information processing device 400 can be connected to the first server device 500 via a network.

The first server device 500 is directed to a server device 500 having a server storage 510 and a server processing unit 520, and preferably an HTTP server using an HTTP protocol, for example.

The server storage 510 receives and stores the biological information transmitted from the information processing terminal 200. The server processing unit generates display information for displaying the biological information on the display screen of the information processing device 400.

This allows a medical worker such as a doctor in a medical institution to check the biological information of the subject by accessing the web page provided by the first server by using the information processing device 400 at hand without requiring to be near the subject.

The biological information displayed on the web page is updated in real time, and in particular, waveform data obtained from an electrocardiograph, for example, is displayed to be continuously checked. However, the deviation of the update in units of several seconds is allowed.

In addition, the information processing terminal 200 can be further connected to the first server device via a second server device (not shown).

The second server device is preferably directed to a message queue telemetrytransport MQTT server using a MQTT technology.

The technology allows the data of the biological information to be transmitted and received at a higher speed with a smaller amount and improves a real-time performance.

<Application Example 1>

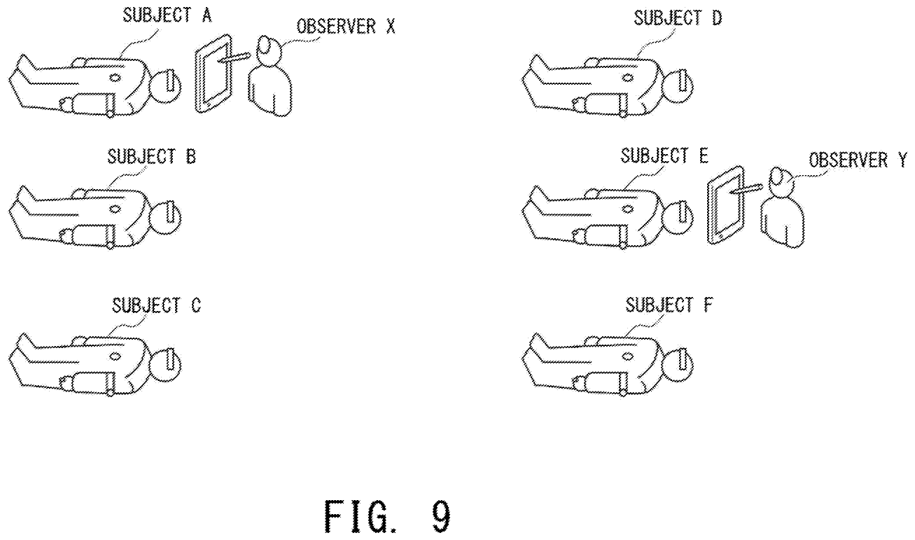

In addition, the present invention has more advantageous effects when used in an emergency, for example, at a disaster site with many subjects. FIG. 9 illustrates an image of a disaster site. When a large number of subjects A to F are within a short distance as shown, the observers X and Y need to observe them without mistaking the information from the measuring devices 100. Even in such a case, the identification information is given to the measuring devices 100 in advance, so that the observers X and Y can select the subject that they wish to observe with the information processing terminal 200 possessed by the observers themselves to check the biological information of the subject.

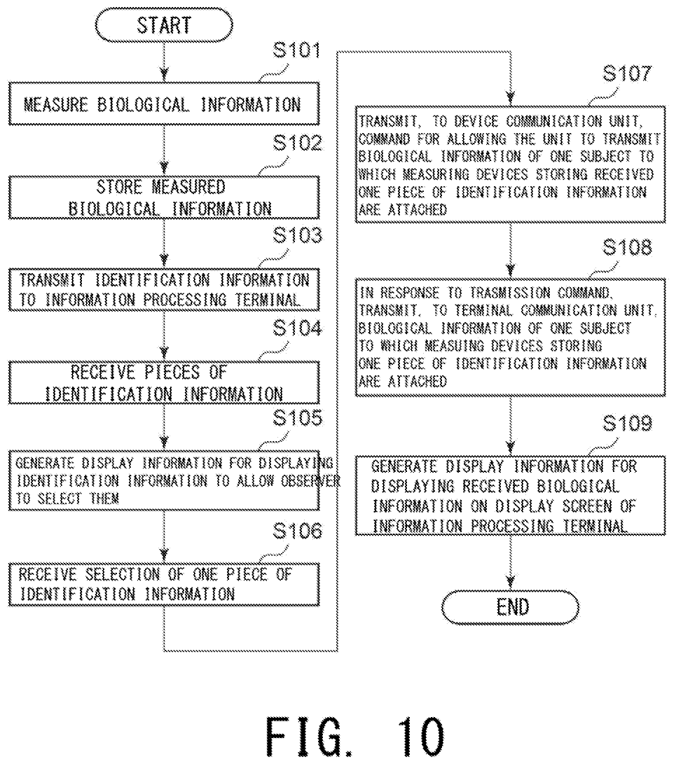

Next, a biological information monitoring method according to an embodiment of the present invention will be described with reference to FIG. 10. FIG. 10 is a flowchart illustrating a flow of a biological information monitoring method according to an embodiment of the present invention.

As shown in FIG. 10, the biological information monitoring method according to an embodiment of the present invention directed to a biological information monitoring method in the biological information monitoring system 1000 including a plurality of measuring devices 100 attached to a plurality of subjects for measuring biological information of the subjects and a portable information processing terminal 200 for an observer for displaying the biological information of the subjects measured by the measuring devices 100 causes the measuring devices 100 to perform a measuring step S101, a device storing step S102, and a deice communicating step S103, and causes the information processing terminal 200 to perform a terminal communicating step S104, a processing step S105, and a selection receiving step S106.

The measuring step S101 measures the biological information of the subject. The measuring step S101 can be performed by the measuring units 110 described above.

The device storing step S102 stores the identification information for identifying the subject and the biological information measured in the measuring step S101. The device storing step S102 can be performed by the device storage 120 described above.

In the device communicating step S103, the identification information stored in the device storing step S102 is transmitted to the information processing terminal 200. The device communicating step S103 can be performed by the device communication unit 130 described above.

The terminal communicating step S104 receives a plurality of pieces of identification information transmitted from the device communication units 130 of the measuring devices 100 attached to a plurality of subjects. The terminal communicating step S104 can be performed by the terminal communication unit 210 described above.

The processing step S105 generates display information for displaying a plurality of pieces of identification information to allow the observer to select them. The processing step S106 can be performed by the processing unit 220 described above.

The selection receiving step S106 receives selection of one piece of identification information from among a plurality of pieces of identification information by the observer. The selection receiving step S106 can be performed by the selection receiving unit 230 described above.

The information processing terminal 200 transmits, to the measuring devices 100, a command for allowing the measuring devices 100 to transmit the biological information of the one subject to which the measuring devices 100 storing the one piece of identification information received in the selection receiving step S106 are attached (step S107).

The measuring devices 100 transmit, to the information processing terminal 200, the biological information of the one subject to which the measuring devices 100 storing the one piece of identification information are attached in response to the transmission command transmitted from the information processing terminal 200 (step S108).

The information processing terminal 200 generates display information for displaying the biological information transmitted from the measuring devices 100 and received by the information processing terminal 200 on the display screen of the information processing terminal 200 (step S109).

The above configuration allows the biological information of a plurality of subjects to be collected and monitored by a simple method.

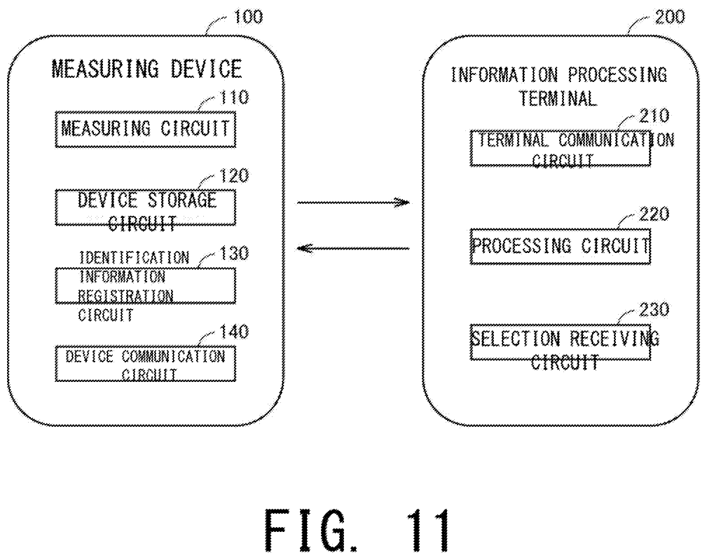

Finally, a biological information monitoring program according to an embodiment of the present invention will be described with reference to FIG. 11. FIG. 11 is a functional configuration diagram illustrating a functional configuration of a biological information monitoring program according to an embodiment of the present invention.

As shown in FIG. 11, the biological information monitoring program according to an embodiment of the present invention directed to a biological information monitoring program for embodying a biological information monitoring system including a plurality of measuring devices 100 attached to a plurality of subjects for measuring the biological information of the subjects and a portable information processing terminal 200 for an observer for displaying the biological information of the subjects measured by the measuring devices 100 causes the information processing terminal 200 to embody a terminal communicating function, a processing function, and a selection receiving function.

The terminal communicating function receives a plurality of pieces of identification information transmitted from a plurality of measuring devices attached to a plurality of subjects. The terminal communicating function can be embodied by a terminal communication circuit 210.

The processing function generates display information for displaying a plurality of pieces of identification information to allow the observer to select them. The processing function can be embodied by a processing circuit 220.

The selection receiving function receives selection of one piece of identification information from among a plurality of pieces of identification information by the observer. The selection receiving function can be embodied by a selection receiving circuit 230.

The terminal communicating function transmits, to the measuring devices, a command for causing the measuring devices to transmit the biological information of one subject to which the measuring devices storing one piece of identification information received by the selection receiving function are attached.

The processing function generates display information for displaying the biological information transmitted from the measuring devices and received by the terminal communication unit on the display screen of the information processing terminal.

The above configuration allows the biological information of a plurality of subjects to be collected and monitored by a simple method.

The above-described computer program according to the embodiments of the present invention may be stored in a processor-readable recording medium, and as the recording medium, a “non-temporary tangible medium” such as a tape, a disk, a card, a semiconductor memory, and a programmable logic circuit may be used, for example.

The computer program can be implemented using, for example, a script language such as ActionScript and JavaScript (registered trademark), an object-oriented programming language such as Objective-C and Java (registered trademark), or a markup language such as HTML5.

### DESCRIPTION OF REFERENCE NUMERALS

1000 . . . biological information monitoring system

100 . . . measuring device

110 . . . measuring unit

120 . . . device storage

130 . . . device communication unit

200 . . . information processing terminal

210 . . . terminal communication unit

220 . . . processing unit

230 . . . selection receiving unit

240 . . . display screen

250 . . . display screen

300 . . . server device

310 . . . server storage

What is claimed is:

1. A biological information monitoring system including a plurality of measuring devices attached to a plurality of subjects for measuring biological information of the subjects, and a portable information processing terminal for an observer for displaying the biological information of the subjects measured by the measuring devices;
each measuring device including:
a measuring unit configured to be managed in advance as a set together with other measuring devices that are attached to one subject, wherein the measuring unit is configured to identify the set that includes the measuring devices, and thereby identification information capable of identifying the subject to which the set of measuring devices are attached is given in advance, and the measuring unit is configured to measure the biological information of the subject,
a device storage configured to store the identification information and the biological information measured by the measuring unit, and
a device communication unit configured to transmit the identification information stored by the device storage to the information processing terminal, the information processing terminal including:
a terminal communication unit configured to receive a plurality of pieces of the identification information transmitted from the device communication unit of each of the measuring devices attached to the subjects,
a processing unit configured to generate display information for displaying the pieces of identification information to allow the observer to select them, and
a selection receiving unit configured to receive a selection of one piece of identification information from the pieces of identification information by the observer, wherein
the terminal communication unit transmits a command to each device communication unit for allowing the device communication unit to transmit biological information of one subject to which the measuring devices storing the one piece of identification information received by the selection receiving unit are attached,
the device communication unit transmits, to the terminal communication unit, the biological information of the one subject to which the measuring devices storing the one piece of identification information are attached in response to the transmission command transmitted from the terminal communication unit, and
the processing unit generates display information for displaying the biological information received by the terminal communication unit on a display screen of the information processing terminal.

2. The biological information monitoring system according to claim 1, wherein information exchange between the measuring devices and the information processing terminal is performed using a BLE wireless communication technology.

3. The biological information monitoring system according to claim 1, wherein the processing unit generates display information for displaying biometric information associated with one piece of identification information associated with one subject selected by the observer within one display screen.

4. The biological information monitoring system according to claim 1, wherein the processing unit generates display information for displaying an instruction button for receiving an instruction from the observer on the display screen, and the instruction button is provided for allowing the device communication unit to transmit, to the terminal communication unit, biometric information of one subject to which the measuring devices storing the one piece of identification information are attached in response to the transmission command transmitted from the terminal communication unit.

5. The biological information monitoring system according to claim 1, wherein the information processing terminal is connected to a server device having a server storage via a network, and the server storage associates the identification information with the subject to which the measuring devices storing the identification information are attached.

6. The biological information monitoring system according to claim 1, wherein the measuring devices are directed to two or more measuring devices selected from an intracerebral oxygen saturation monitor, an electrocardiograph, a sphygmomanometer, a pulse oximeter, and an echo.

7. A biological information monitoring system including a plurality of measuring devices attached to a plurality of subjects for measuring biological information of the subjects, and a portable information processing terminal for an observer for displaying the biological information of the subjects measured by the measuring devices;
each measuring device including:
a measuring unit configured to be managed in advance as a set together with other measuring devices that are attached to one subject, and measure biological information of the subject,
a device storage configured to store the identification information given for each set of the measuring devices, and the biological information measured by the measuring unit,
an identification information generator configured to issue additional information in response to a request, generate identification information including the additional information and the identification information as identification information of one subject to which the measuring devices included in one set are attached, and allow the device storage to store the identification information, and
a device communication unit configured to transmit the identification information of the subject stored by the device storage to the information processing terminal,
the information processing terminal including:
a terminal communication unit configured to receive a plurality of pieces of the identification information of the subjects transmitted from the device communication unit of each of the measuring devices attached to the subjects,
a processing unit configured to generate display information for displaying the pieces of identification information of the subjects to allow the observer to select them, and
a selection receiving unit configured to receive a selection of one piece of identification information of one subject from the pieces of identification information of the subjects by the observer, wherein
the terminal communication unit transmits a command to each device communication unit for allowing the device communication unit to transmit biological information of the one subject to which the measuring devices storing the one piece of identification information of the one subject received by the selection receiving unit are attached,
the device communication unit transmits, to the terminal communication unit, the biological information of the one subject to which the measuring devices storing the one piece of identification information of the one subject are attached in response to the transmission command transmitted from the terminal communication unit, and
the processing unit generates display information for displaying the biological information received by the terminal communication unit on a display screen of the information processing terminal.

8. The biological information monitoring system according to claim 7, wherein the identification information generator uses time information or position information as the additional information.

9. The biological information monitoring system according to claim 7, wherein the information processing terminal further includes:
a subject information receiving unit that receives subject information of the subject acquired from the subject by the observer, and
a subject identification unit that identifies one subject having subject information corresponding to the subject information received by the subject information receiving unit from a subject information database stored in advance in a predetermined storage device, wherein
the terminal communication unit transmits an instruction to the device communication unit for allowing the device communication unit to transmit biological information of the one subject to which the measuring devices storing the one piece of identification information received by the selection receiving unit are attached, or biological information of the one subject identified by the subject identifying unit.

10. The biological information monitoring system according to claim 9, wherein the subject information includes at least one of name information, fingerprint information, vein information, face information, and/or iris information.

11. The biological information monitoring system according to claim 7, wherein the biological information monitoring system further includes an information processing device for remotely monitoring the biological information,
the information processing terminal and the information processing device are connected via a network to a first server device having a server storage and a server processing unit,
the server storage receives and stores the biological information transmitted from the information processing terminal, and
the server processing unit generates display information for displaying the biological information on a display screen of the information processing device.

12. The biological information monitoring system according to claim 11, wherein the information processing terminal is further connected to the first server device via a second server device as an MQTT server.

13. A biological information monitoring method performed in a biological information monitoring system including a plurality of measuring devices attached to a plurality of subjects for measuring biological information of the subjects, and a portable information processing terminal for an observer for displaying the biological information of the subjects measured by the measuring devices,
the method causing each of the measuring devices to perform:
a measuring step of being managed in advance as a set together with other measuring devices that are attached to one subject, wherein the measuring device is configured to identify the set that includes the measuring devices, and thereby identification information capable of identifying the subject to which the set of measuring devices are attached is given in advance, and the measuring devices are configured to measure the biological information of the subject,
a device storing step of storing the identification information and the biological information measured in the measuring step, and
a device communicating step of transmitting the identification information stored in the device storing step to the information processing terminal,
the method further causing the information processing terminal to perform:
a terminal communicating step of receiving a plurality of pieces of the identification information transmitted from a device communication unit of each of the measuring devices attached to the subjects,
a processing step of generating display information for displaying the pieces of identification information to allow the observer to select them, and
a selection receiving step of receiving a selection of one piece of identification information from the pieces of identification information by the observer, wherein
the information processing terminal transmits a command to the measuring devices for allowing the measuring devices to transmit biological information of one subject to which the measuring devices storing the one piece of identification information received in the selection receiving step are attached,
the measuring devices transmit, to the information processing terminal, the biological information of the one subject to which the measuring devices storing the one piece of identification information are attached in response to the transmission command transmitted from the information processing terminal, and
the information processing terminal generates display information for displaying the biological information transmitted by the measuring devices and received by the information processing terminal on a display screen of the information processing terminal.

14. A biological information monitoring method performed in a biological information monitoring system including a plurality of measuring devices attached to a plurality of subjects for measuring biological information of the subjects, and a portable information processing terminal for an observer for displaying the biological information of the subjects measured by the measuring devices,
the method causing the measuring devices to perform:
a measuring step of being managed in advance as a set together with other measuring devices that are attached to one subject, wherein identification information is given to each measuring device in advance for each set of the measuring devices, and measuring the biological information of the subject,
an identification information generating step of issuing additional information in response to a request, and generating identification information including the additional information and the identification information as identification information of one subject to which the measuring devices included in one set are attached,
a device storing step of storing the identification information of the subject and the biological information measured in the measuring step, and
a device communicating step of transmitting the identification information of the subject stored in the device storing step to the information processing terminal,
the method further causing the information processing terminal to perform:
a terminal communicating step of receiving a plurality of pieces of the identification information of the subjects transmitted from a device communication unit of each of the measuring devices attached to the subjects,
a processing step of generating display information for displaying the pieces of identification information of the subjects to allow the observer to select them, and
a selection receiving step of receiving a selection of one piece of identification information of one subject from the pieces of identification information of the subjects by the observer, wherein
the information processing terminal transmits a command to the measuring devices for allowing the measuring devices to transmit biological information of one subject to which the measuring devices storing the one piece of identification information of the one subject received in the selection receiving step are attached,
the measuring devices transmit, to the information processing terminal, the biological information of the one subject to which the measuring devices storing the one piece of identification information are attached in response to the transmission command transmitted from the information processing terminal, and
the information processing terminal generates display information for displaying the biological information transmitted by the measuring devices and received by the information processing terminal on a display screen of the information processing terminal.

15. A biological information monitoring program for embodying a biological information monitoring system including a plurality of measuring devices attached to a plurality of subjects for measuring biological information of the subjects, and a portable information processing terminal for an observer for displaying the biological information of the subjects measured by the measuring devices,
the program causing each of the measuring devices to embody:
a terminal communicating function of managing each measuring device in advance as a set together with other measuring devices that are attached to one subject, wherein the measuring device is configured to identify the set that includes the measuring devices, and thereby identification information capable of identifying the subject to which the set of measuring devices are attached is given in advance, and causing the information processing terminal to receive a plurality of pieces of the identification information transmitted from the measuring devices attached to the subjects,
a processing function of generating display information for displaying the pieces of identification information to allow the observer to select them, and
a selection receiving function of receiving a selection of one piece of identification information from the pieces of identification information by the observer, wherein
the terminal communicating function transmits a command to the measuring devices for allowing the measuring devices to transmit biological information of one subject to which the measuring devices storing the one piece of identification information received in the selection receiving function are attached, and
the processing function generates display information for displaying the biological information transmitted by the measuring devices and received by the terminal communicating function on a display screen of the information processing terminal.

16. A biological information monitoring program for embodying a biological information monitoring system including a plurality of measuring devices attached to a plurality of subjects for measuring biological information of the subjects, and a portable information processing terminal for an observer for displaying the biological information of the subjects measured by the measuring devices,
the program causing each of the measuring devices to embody:
a terminal communicating function of managing each measuring device in advance as a set together with other measuring devices that are attached to one subject, wherein identification information is given for each set of the measuring devices in advance, and causing the information processing terminal to receive a plurality of pieces of the identification information of the subjects transmitted from the measuring devices attached to the subjects,
a processing function of generating display information for displaying the pieces of identification information of the subjects to allow the observer to select them, and
a selection receiving function of receiving a selection of one piece of identification information of the one subject from the pieces of identification information of the subjects by the observer, wherein
the terminal communicating function transmits a command to the measuring devices for allowing the measuring devices to transmit biological information of the one subject to which the measuring devices storing the one piece of identification information of the one subject received in the selection receiving function are attached, and
the processing function generates display information for displaying the biological information transmitted by the measuring devices and received by the terminal communicating function on a display screen of the information processing terminal.
